(19) United States
(12) Patent Application Publication
(10) Pub. No.: US 2026/0207213 A1
(43) Pub. Date: Jul. 23, 2026

(54) OPHTHALMIC ARTERY ANGIOPLASTY BALLOON AND GUIDEWIRE BASED INTRA VASCULAR LITHOTRIPSY (IVL) FOR TREATMENT OF OPHTHALMIC DISEASES

(71) Applicant: J.D. Franco & Co., LLC, Plano, TX (US)

(72) Inventors: Michael CALHOUN, Lighthouse Point, FL (US); Jeffrey FRANCO, Plano, TX (US)

(73) Assignee: J.D. Franco & Co., LLC, Plano, TX (US)

(21) Appl. No.: 19/571,082

(22) Filed: Mar. 18, 2026

Related U.S. Application Data
(63) Continuation of application No. PCT/US24/47377, filed on Sep. 19, 2024.
(60) Provisional application No. 63/583,885, filed on Sep. 20, 2023.

(51) Int. Cl. A61B 17/22; A61B 17/00; A61B 90/00

(52) U.S. Cl. CPC A61B 17/22022; A61B 17/00234; A61B 90/39; A61B 2017/00305; A61B 2017/00778; A61B 2017/22025; A61B 2017/22038; A61B 2017/22051; A61B 2090/3966

(57) ABSTRACT

An intra vascular lithotripsy (IVL) device for treatment of lesions and/or calcifications within vasculature behind an eye of a subject, the IVL device may include a guidewire configured to access the vasculature of the subject; and a balloon catheter including: an inflatable balloon configured to be placed within a treatment area of the vasculature of the subject and to be inflated therein, and an electrode arrangement within the balloon, wherein, the electrode arrangement is configured to deliver one or more shock waves, and to the treatment area, when electrical energy is supplied to the electrode arrangement.

### CROSS-REFERENCE TO RELATED APPLICATIONS

This application is a continuation application of and claims the benefit of priority to PCT Application No. PCT/US2024/047377, filed on Sep. 19, 2024, which claims the benefit of priority to U.S. Provisional Patent Application No. 63/583,885, filed on Sep. 20, 2023, which are incorporated herein in their entireties.

### FIELD OF INVENTION

The present disclosure relates to intravascular devices, specifically, intra vascular lithotripsy (IVL devices including balloons and guidewires, for the treatment of vascular obstructions in the vasculature behind the eye.

### BACKGROUND

Intravascular lithotripsy (IVL) is a concept in which catheter-based devices are used to modify calcified lesions via delivery of acoustic shock waves. IVL capability may be incorporated into angioplasty balloons or guidewires (GW). The concept uses acoustic shock waves to reduce the homogeneity of a calcified lesion by fracturing it into smaller pieces. Fracturing allows for more effective angioplasty, including stent deployment, with a reduced risk of injury to the treated artery. IVL is based on the concept of extracorporeal shockwave therapy in which various types of mineralized stones are broken up within the body so they may be passed out naturally without surgical intervention. These areas of the body include kidneys, gallbladder, liver, pancreas, and salivary glands.

Generally, lesions may be within the lumen or wall of the artery to be treated.

Intravascular treatment involves angioplasty in which a balloon dilatation catheter and/or a GW is used to cross a lesion in order to dilate and restore normal or near normal blood flow in the artery. Angioplasty is most effective when the lesion is a calcified within the wall of the artery, but it is not limited to this condition and may be used on intra luminal lesions as well. There are several types of lesions which may benefit from angioplasty. These include total occlusions, concentric and non-concentric calcified lesions. Calcified lesions may require balloon inflation pressures from 10 to 15 atmospheres. In some peripheral and coronary cases, higher pressures are needed to break the calcified plaque and push it back into the vessel wall. These inflation pressures introduce the risk of trauma to the vessel wall which can cause vessel rebound, dissection, and thrombus formation, and may contribute to restenosis of the treated segment. Once the balloon is placed within the lesion, it is filled with fluid and pressure is increased to inflate the balloon and open the lumen to re-establish or improve blood flow. As the balloon is inflated, it is confined to the diameter of the calcified lesion. As the balloon inflation pressure increases, a large amount of energy is stored in the balloon until the calcified lesion breaks or cracks. When that happens, the stored energy in the balloon is suddenly released and a rapid expansion of the balloon to its maximum diameter occurs. If this expansion is not controlled, this sudden change in diameter may injure the vessel walls. IVL is used to pretreat the lesion so that the calcification is fractured to reduce the chance of the lesion storing energy during angioplasty.

Until recently, angioplasty treatment of the OA for eye disease was not contemplated, as there were no ocular disorders associated with diseased OA. In addition, the OA was considered to be neural tissue and, therefore, it was thought to be very fragile, so angioplasty treatment was avoided. Recent publications indicate that there is disease present in the OA that may affect certain ocular conditions. Publications have also demonstrated that angioplasty treatment of the OA has been shown to improve visual acuity in certain patient groups and this treatment was well tolerated. Combining IVL with balloon angioplasty and GWs for treatment of the OA has not previously been contemplated but may offer the ability to treat calcific lesions as described to improve angioplasty outcomes.

There is also a need to minimize the potential for embolic events in the retinal circulation as a result of OA angioplasty. As noted above, the retina is extremely sensitive to any interruption in blood flow, and devastating visual damage can result. While there is no published information on embolic complication rates for angioplasty performed in the OA, previous literature indicates that neurological complication rates for carotid angioplasty and stenting without distal protection may range from 3.3% to 10.9% due to distal embolization. To reduce the likelihood of this event, distal protection devices have been used in these procedures. The purpose of these devices is to capture and remove debris generated from angioplasty before it can travel downstream and cause complications. Currently there are several types of devices used in cerebral procedures for angioplasty related embolic protection. These types of devices broadly include 1) flow preservation devices also called distal filters, 2) distal occlusion devices, and 3) proximal protection devices. It is worth noting that none of these devices have been contemplated for use in the OA.

The invention according to the present disclosure is focused on IVL therapy delivered to the ophthalmic artery (OA) via balloon angioplasty and/or GWs for the treatment of ophthalmic diseases. The inventions described herein provide IVL devices, which may include a balloon catheter, a GW, or both, for treatment of obstructions (e.g., stenosis, lesions, and plaques) within an intravascular lumen or within the walls of a vascular structure for the purpose of treating eye disease. One example of use of this system in the OA is for the treatment of age-related macular degeneration (AMD).

### SUMMARY

In one aspect, an intra vascular lithotripsy (IVL) device for treatment of lesions and/or calcifications within vasculature behind an eye of a subject may include a guidewire configured to access the vasculature of the subject; and a balloon catheter including: an inflatable balloon configured to be placed within a treatment area of the vasculature of the subject and to be inflated therein, and an electrode arrangement within the balloon, wherein, the electrode arrangement is configured to deliver one or more shock waves, and to the treatment area, when electrical energy is supplied to the electrode arrangement.

In another aspect, a method of treating lesions and/or calcifications within vasculature behind an eye of a subject using an intra vascular lithotripsy (IVL) device is described. The IVL device may include a guidewire for accessing vasculature of the subject, a balloon catheter including an inflatable balloon configured to be placed within a treatment area of the vasculature of the subject and to be inflated therein, and an electrode arrangement within the balloon, and the method may include accessing an ophthalmic artery (OA) of the subject using the guidewire; placing the balloon within a treatment area near an internal carotid artery (ICA) or within the OA; inflating the balloon a first time within the treatment area; and supplying electrical energy to the electrode arrangement, thereby initiating and delivering one or more shock waves to the treatment area.

In another aspect, an intra vascular lithotripsy (IVL) device for treatment of lesions and/or calcifications within vasculature behind an eye of a subject may include a hypotube; a guidewire configured to access the vasculature of the subject, a proximal portion of the guidewire being housed within the hypotube; and an electrode arrangement within the guidewire, wherein, the electrode arrangement is configured to deliver one or more shock waves, through the guidewire and the hypotube, and to a treatment area, when electrical energy is supplied to the electrode arrangement.

### BRIEF DESCRIPTION OF THE DRAWINGS

FIG. 1 is a digital image of vasculature behind an eye of a human subject, and in particular, shows an ophthalmic artery (OA) and an internal carotid artery (ICA).

FIG. 2A is a digital image depicting the vasculature of a subject without age-related macular degeneration (AMD), and FIG. 2B is a digital image depicting the vasculature of a subject with AMD.

FIG. 3A is a digital image showing histopathology of a normal OA as it branches from the ICA, FIG. 3B is a digital image showing histopathology of an OA blocked at the ostium of the OA and the ICA, and FIG. 3C is a digital image showing histopathology of medial calcifications in the short limb (SL) of the OA.

FIG. 4A is a digital image of an OA, and FIG. 4B is a schematic detail of a takeoff angle of the OA from the ICA.

FIG. 5 is a digital image depicting the distance from the origin of the OA to the CRA branch.

FIG. 6 a schematic diagram showing the OA origin from the ICA

FIGS. 7A, 7B, and 7C show an IVL device, which may be configured for use within the SL of the OA, according to one embodiment.

FIGS. 8A, 8B, and 8C show an IVL device, which may be configured for use in the SL of the OA, according to another embodiment.

FIGS. 9A, 9B, and 9C show an IVL device, which may be configured for use in a segment of the OA other than the SL, according to yet another embodiment.

FIGS. 10A, 10B, and 10C show an IVL device, which may be configured for use in a non-SL segment of the OA, according to still another embodiment.

FIGS. 11A, 11B, and 11C show an IVL device, which may be configured for use within the ICA for treating a lesion of the OA ostium, according to another embodiment.

FIG. 12 is a flowchart of a method of using an IVL device, according to one or more embodiments.

FIG. 13 is a Cone Beam Computed Tomography (CB CT) image of a calcified OA SL lesion.

FIG. 14 is a flowchart of another embodiment of a method of use of an IVL device, according to another embodiment.

FIG. 15 is a Digital Subtraction Angiography (DSA) image of a non-concentric OA SL lesion.

FIG. 16 is a schematic of an IVL device, according to one embodiment.

FIG. 17 is a flowchart of a method of using an IVL device, according to another embodiment.

FIG. 18 is a flowchart of a method of using an IVL device, according to still another embodiment.

FIG. 19 is a flowchart of a method of using IVL device, according to yet another embodiment.

FIG. 20 is a flowchart of a method of using an IVL device, according to another embodiment.

FIGS. 21A and 21B show an IVL device, according to an embodiment.

FIGS. 22A and 22B show an IVL device, according to another embodiment.

FIG. 23 shows an end of an electrode of an IVL device with a coating thereon, according to one or more embodiments.

FIGS. 24A and 24B show an IVL device having a distal filter, according to one or more embodiments.

FIGS. 25A and 25B show an IVL device having a distal occlusion balloon, according to one or more embodiments.

### DETAILED DESCRIPTION

Various embodiments of the present disclosure relate generally to devices for use in intra vascular lithotripsy (IVL) procedures and related methods of using the same.

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass±10% of a specified amount or value. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more. As used herein, the terms “comprises,” “comprising,” “including,” “having,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” In addition, the term “between” used in describing ranges of values is intended to include the minimum and maximum values described herein. The term “proximal” is used to describe the end of a device that is located closest to an operator of the device when using a device on a subject, whereas the term “distal” is used to describe the end of a device that is located closest to a subject on whom the device is being used and farthest away from the operator.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

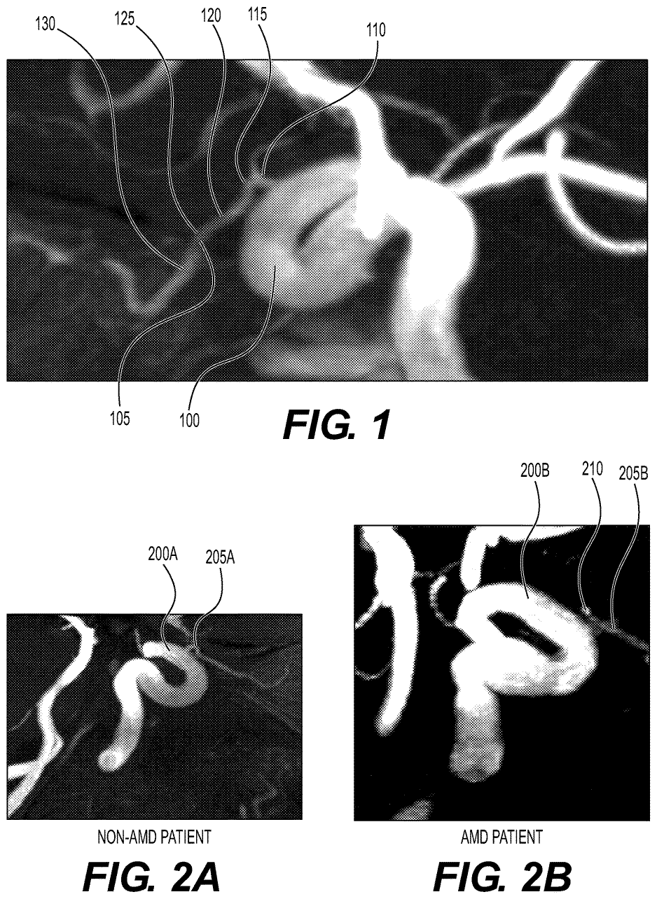

With reference to FIG. 1, the anatomy of the vasculature behind an eye of a human subject (not shown) will be described. In particular, FIG. 1 is a digital image showing the internal carotid artery (ICA) 100, and the ophthalmic artery (OA) 105, including the short limb 110 of the OA 105, an angle ‘a’ 115 of the OA 105, a long limb 120 of the OA 105, an angle ‘b’ 125 of the OA 105, and a distal part 130 of the OA 105. The OA 105 is an autoregulating, terminal branch of the ICA 100, and provides the majority supply of blood to the eye. FIG. 1 shows the OA 105 as a branch of the ICA 100, and depicts the arrangement of the OA 105, including the arrangement of the short limb 110, the angle ‘a’ 115, the long limb 120, the angle ‘b’ 125, and the distal part 130 of the OA 105, as it branches from the ICA 100 in a non-diseased vessel. The retina (not shown) is supplied with oxygenated blood by the OA 105, and rests in the back of the eye. It contains approximately 126 million photoreceptors. These photoreceptors consume more oxygen than any other tissue in the human body. Any disruption or blockage (i.e., interruption) of normal blood flow rates to the eye impacts the flow of nutrients into and the flow of waste products out of the eye. This flow alteration may cause a lack of oxygen flow (via blood flow) to the photoreceptors and allow the accumulation of waste products. These conditions may cause devastating injury to photoreceptors and in turn have a negative effect on vision. In some cases, irreversible vision damage may result.

FIG. 2A is a digital image depicting the vasculature of a subject without age-related macular degeneration (AMD), including an ICA 200A and an OA 205A, and FIG. 2B is a digital image depicting the vasculature of a subject with AMD, with an ICA 200B and an OA 205B. Note the OA 205B of the subject with AMD, shown in FIG. 2B, is blocked by stenosis 210.

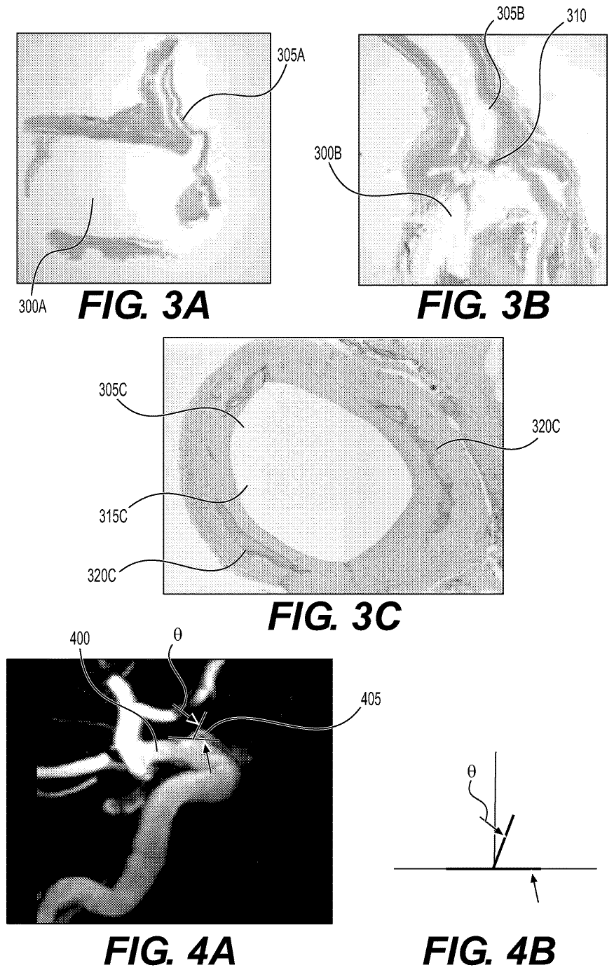

FIG. 3A is a digital image depicting histopathology of a normal OA 305A as it branches from an ICA 300A, FIG. 3B is a digital image depicting histopathology of an OA 305B that is blocked at the ostium of the OA 305B and an ICA 300B by a lesion 310 (also referred to as a total occlusion OA), and FIG. 3C is a digital image depicting histopathology of a short limb 315C of an OA 305C, with medial calcifications 320C. The vasculature of FIGS. 3B and 3C would be IVL treatment targets.

Treatment of the OA, such as OA 205B in FIG. 2B, the OA 305B in FIG. 3B, or the OA 305C in the FIG. 3C, may be indicated in cases where luminal stenosis is 50% or less. In one example method of use of the IVL devices of the present disclosure, including a balloon catheter and/or a guidewire (GW), OA lesions in the range of 20% to 40% stenosis may be treated. It has not been previously contemplated that treatment of lesions with less than 50% stenosis would be meaningful. As noted above, the photoreceptors of the retina are the most metabolically active tissue in the human body, and even a luminal stenosis of less than 50% can have a devastating effect on the photoreceptors.

Table 1 lists examples of OAs, and specifically, OAs of specific diameters, cross-sectional areas thereof, percentage decrease in cross-sectional areas, and percentage stenosis based on the percentage decrease in cross-sectional areas. In particular, in example OA number 2, a 16% stenosis has an impact equivalent to a 30% reduction in cross-sectional blood flow area. Further, in example OA number 4, a 50% stenosis has a 75% reduction in cross-sectional blood flow area. At least these two example OAs show that a luminal stenosis of 50% or less can have an impact equivalent to a percentage decrease in cross-sectional area of, for example, 30.56%, 55.56%, or 75%, and, therefore, a luminal stenosis of 50% or less can significantly affect blood supply to the photoreceptors.

TABLE 1

| | OA | Cross-Sectional | Percentage | |
|---|---|---|---|---|
| | Diameter | Area (CSA) | Decrease in | Percentage |
| Example OA # | (mm) | (mm²) | CSA (mm²) | Stenosis |
| 1 | 1.20 | 1.1310 | 0.00 | 0.00 |
| 2 | 1.00 | 0.7854 | 30.56 | 16.67 |
| 3 | 0.80 | 0.5027 | 55.56 | 33.33 |
| 4 | 0.60 | 0.2827 | 75.00 | 50.00 |
| 5 | 0.40 | 0.1257 | 88.89 | 66.66 |
| 6 | 0.20 | 0.0314 | 97.22 | 83.33 |
| 7 | 0.00 | 0.0000 | 100.00 | 100.00 |

In addition to not appreciating lower levels (percentages) of stenosis having significant effect on blood flow through small diameter arteries, there is also a lack of appreciation for the complexity of the OA anatomy and the degree of difficulty in accessing and treating the OA anatomy using conventional tools. The most challenging aspect of this treatment is accessing the desired treatment location within the OA. Current IVL devices are not designed for use in this area (that is, in the vasculature behind the eye), and do not address this unique anatomy, which is smaller in diameter, and which is angulated and significantly more tortuous, as compared to typical cardiac vessels.

FIG. 4A is a digital image of an OA 405 branching off of an ICA 400, and, in particular, depicts a takeoff angle θ, at the origin of the OA 405, which contributes to the difficulty in accessing the OA 405, as the OA 405 branches from or takes off from the ICA 400. The takeoff angle θ, shown in detail in FIG. 4B, may be about 78°. The IVL devices described herein are designed and configured to access OA ostiums with takeoff angles (that is, angles between the ICA and the OA branching therefrom) from about 45° to about 140°, and to be placed within portions of the OA between the ostium down to the central retinal artery.

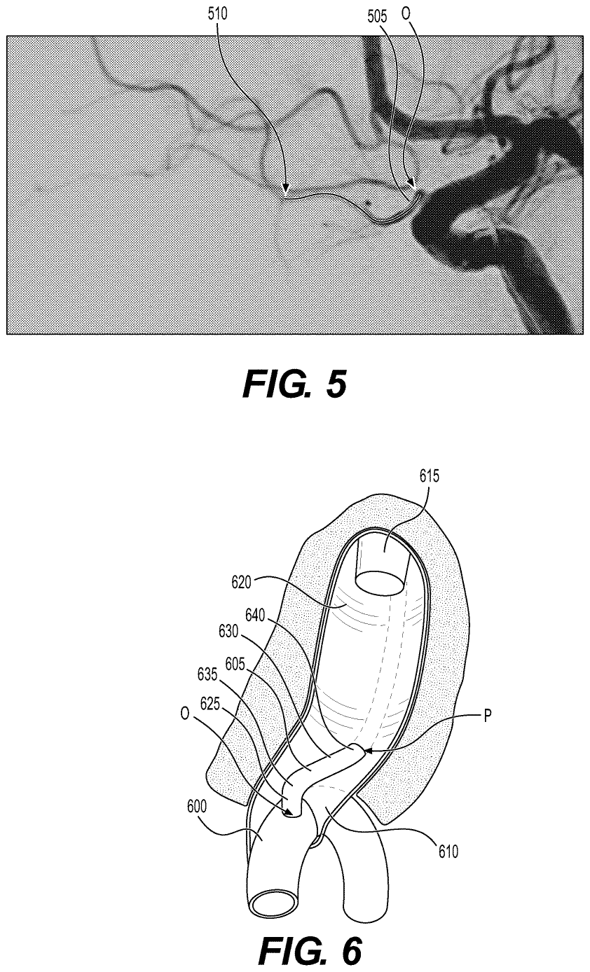

The IVL devices described herein are configured for access and placement within the OA, and to have relatively lower bulk and stiffness as compared to that of devices used for cardiac and other applications. Further, the IVL devices described herein are configured for treatment of the OA. The diameter of the OA as it branches off the ICA at an angle, also referred to as the origin diameter, may range from about 1.2 mm to about 2 mm in a healthy human subject. Portions of the OA closer to the eye (in other words, more distal portions of the OA) typically have reduced diameters, which may be less than about 1 mm along the segment from the origin of the OA to the branch of the central retinal artery. In the example of treatment of the OA, the target area includes the OA origin at the ICA and continues distally (that is, towards the eye), until the approximate branch of the central retinal artery (that is, the CRA branch) from the OA. This total length may be in the range of about 10 mm to about 25 mm. FIG. 5 is a digital image depicting the length from the origin O of the OA 505 to the CRA branch 510, which may be approximately 21.4 mms. It may be desirable to treat anywhere along this portion or length of the OA 505 with the devices and methods described herein.

An area of particular interest for IVL treatment is the SL of the OA. As noted above, this is the initial branch of the OA off the ICA. The SL may be the site of total occlusions and/or concentric or non-concentric calcified lesions. To exert sufficient force to break the calcified lesions and push portions of broken lesions back into the vessel wall, balloons may be required to be inflated to pressures (balloon inflation pressures) from about 8 atmospheres (ATMs) to about 15 ATMs. Use of IVL devices and methods in these locations fragments calcified lesions, and may reduce the risk of subsequent balloon angioplasty damage to the vessel. FIG. 6 is a schematic diagram showing an origin O of an OA 605 from an ICA 600, and more particularly, shows the ICA 600, a periosteum 610, a point P of penetration of a dural sheath (not shown), the OA 605, an optic nerve 615, an optic canal 620, an SL 625, a long limb 630 of the OA 605, angle ‘a’ 635 between the ICA 600 and the SL 625, and angle ‘b’ 640 between the SL 625 and the LL 630. Typical SL lengths range from about 0.7 mm to about 2.7 mm.

An IVL device may include either a catheter having a balloon, such as an angioplasty balloon, at the distal end and configured to be inflated with a fluid, or a catheter having a GW, or both either of which is to be placed within the OA. Disposed within the balloon or GW is an electrode arrangement, connected to a shock wave generator, and which may be either a bipolar or unipolar electrode arrangement. The particular electrode arrangement is dependent on the need and is coupled to a high voltage source, via the shock wave generator, at the proximal end of the catheter or GW through a connector. When the balloon or GW is placed adjacent to a calcified region of an artery (also referred to as a target), and one or more high voltage pulses are supplied across the electrodes, one or more shock waves may be formed that propagate through the fluid (blood or saline), and impinge upon the calcified region. Repeated pulses break up the calcium (i.e., the calcified region) without damaging surrounding soft tissue. In embodiments in which the device includes a balloon, the shock wave(s) may be transmitted first through the fluid within the balloon, and then to the target anatomy or treatment area. In embodiments that include a GW, the shock wave(s) may be transmitted through the blood to the target anatomy or treatment area. In some cases, there is also a provision for the GW to contain saline.

According to the present disclosure, in one embodiment, an IVL device may include a balloon catheter, including a balloon, and an electrode arrangement, a shock wave generator, and a power source. The electrode may be conventional or low profile with an electrode design that is mono polar or bipolar and may be constructed to provide energy delivery to a particular segment of the artery (i.e., a specific length of the artery) and to an entire inner surface of that segment of the artery (i.e., 360° circumferential) or to a portion thereof. The balloon catheter may be configured to provide the necessary minimal crossing profile, flexibility, torqueability and pushability to access the OA in conjunction with, in some embodiments, use of an appropriately sized GW and/or a microcatheter. The balloon catheter may also be configured to fit within the diameter of the OA artery, as noted above, and to deliver one or more therapeutic shock waves to the target anatomy or treatment area.

The voltage, current, temperature, time, number of pulses, and treatment algorithm ranges of the IVL device are optimized to treat the small diameter of the OA. The treatment algorithm provides information related to how effective (fractured) the shock waves have been to the lesion. The treatment algorithm may account for one or more of voltage levels, current levels, temperature, time, number of pulses, IVL device placement and angioplasty balloon placement, and balloon size and balloon inflation time, as parameters. Before treatment with the IVL device and angioplasty (pre-treatment), the vessel segment is imaged, and, when the treatment with the IVL device and angioplasty are completed (post-treatment), the vessel segment is imaged again. One or more measurements of the post-treatment image are compared to corresponding measurement(s) of the pre-treatment image to determine results of the treatment and assess whether they meet an acceptable standard. For example, a measurement of a lumen (i.e., a luminal measurement, such as a measurement of a diameter of a lumen) of the vessel segment is obtained from each of the pre-treatment image and the post-treatment image, the luminal measurements are compared to each other, and a difference in luminal measurement from pre-treatment and post-treatment is compared to a predetermined value to determine if the treatment with the IVL device angioplasty treatment increased the vessel lumen. In other words, if the different in luminal measurement is greater than 0 mm, the result may be that the treatment meets an acceptable standard as it increased the luminal measurement. The one or more measurements, including these luminal measurements, may be taken by use of digital selective angiography (DSA), mean aneurysm flowrate analysis (MAFA), perfusion measurement, or any means typically used to assess changes in luminal diameter as a result of IVL treatment and/or angioplasty. If the results are not acceptable (for example, if the difference in luminal diameter is 0 mm), then the parameters may be adjusted, and a subsequent treatment may be performed. The process may be repeated until a measurement, such as a luminal measurement, meets a predetermined measurement which may be indicative of successful treatment such that treatment may end or conclude. Over time, the data relating to the one or more measurements is fed into the algorithm to generate recommended values for the parameters, and, in turn, to provide procedural recommendations.

The shock waves may include forward directed waves, radially directed waves, circumferentially directed waves, any partially arc directed waves, or any combination of directions or waves. In addition, the IVL device may have a non-cavitation design to provide therapeutic shock wave energy within a very small diameter artery. Non-cavitation design means that the IVL device uses the acoustic property of the traveling shock wave(s) to impact the target, without the need to generate bubbles. In this example, the shock wave(s) would not travel through a liquid medium (i.e., saline or blood), but would travel directly from the IVL device to the target (that is, the shock wave(s) travel through air within the balloon). In some embodiments, however, the IVL device may be configured for filling of the balloon with a liquid medium (e.g., saline or blood), such that the shock wave(s) do travel through the liquid medium and such that air bubbles within the liquid are generated and used to provide the therapeutic shock wave energy within an artery.

The balloon may be configured to be extremely flexible with a balloon compliancy profile that may be compliant, semi-compliant, or non-compliant. In some embodiments, the balloon may have a combination of compliance along the length of the balloon. The crossing profile, defined as a maximum diameter between a proximal end of the balloon and a distal tip of the catheter, is about 0.035 inches (0.889 mm) or less, so as to fit within the lumen of the OA, and, in particular, to fit within a lumen of a diseased or occluded OA. The balloon may have an inflated diameter (that is, inflated balloon diameter) in the range of about 0.75 mm to about 2.5 mm, and this diameter may be selected or designed to provide optimized angioplasty to the OA. In one embodiment, the inflated balloon diameter is about 1.5 mm. Inflated balloon diameters in this range allow for a balloon to artery ratio (B/A) (that is, a ratio of inflated balloon diameter to artery diameter) in a range of about 0.9 to about 1.3, which may be the most desirable range for maintaining a balance between lumen patency and procedural safety. Inflation pressures of the balloon may be in a range of about 4 ATMs to about 8 ATMS. In some instances, a maximum balloon inflation pressure may be about 12 ATMs. A length of the balloon when inflated (that is, an inflated balloon length) may be in a range of about 4 mm to about 30 mm, and may be chosen based on whether a lesion is located in the SL of the OA, or further down in the OA, as well as whether the lesion is focal or diffuse. Balloons contain electrode arrangements, and thus, the inflated balloon length may also be chosen to specially address energy delivery to specific segments of the OA (in terms of length and radial location of the target anatomy).

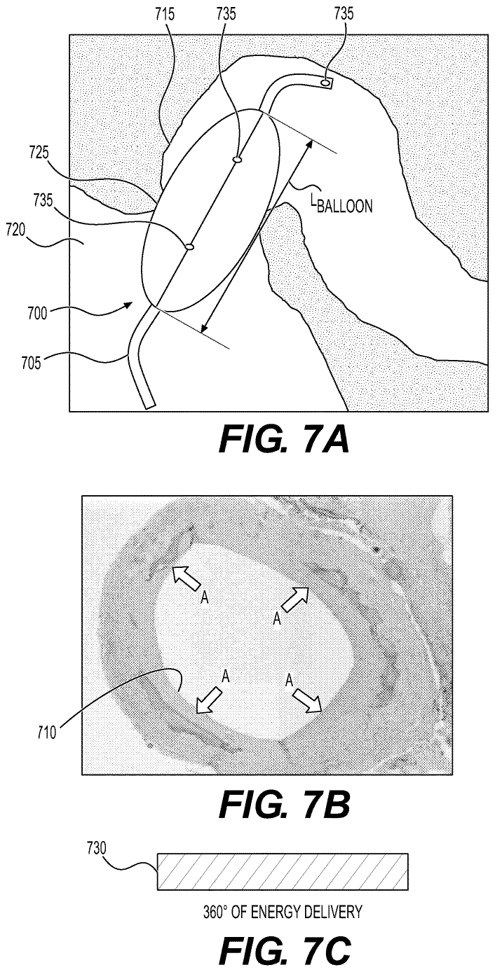

FIGS. 7A, 7B, and 7C show an IVL device 700 according to one embodiment, having a balloon catheter 705, which may be configured for use within an SL 710 of an OA 715, according to one embodiment. In FIG. 7A, the balloon catheter 705 is shown positioned within the SL 710 of the OA 715, which branches off of an ICA 720. The balloon catheter 705 may have a balloon 725 having a length L_{BALLOON} designed for inflation only in the SL 710 of the OA 715. FIG. 7B shows a cross-sectional view of the SL 710 of the OA 715, as well as arrows A indicating the directions in which the electrical energy from an electrode arrangement 730, shown in FIG. 7C, is delivered to the SL 710. The four arrows A in FIG. 7B demonstrate the multi-directional delivery of the electrical energy, as shock waves, and, more specifically, the delivery of the shock waves to a 360° circumferential area of the SL 710. FIG. 7C is a schematic showing electrical supply from the electrode arrangement 730 of the IVL device 700 to a 360° circumferential area. That is, the balloon catheter 705 of this embodiment is configured to deliver one or more shock waves specifically to the SL 710 in a 360° circumferential area, while preventing energy from affecting surrounding tissue, such as the ICA 720 or tissue further down the OA 715. The balloon catheter 705 of this embodiment may include radiopaque markers 735 within the balloon 725 and at a distal end of the balloon catheter 705, as shown in FIG. 7A, which may allow a user (e.g., a surgeon or a physician) to properly orient and position the balloon catheter 705 and the balloon 725 prior to providing electrical energy, as part of a method of treatment. In particular, at least one of the radiopaque markers 735 within the balloon 725 may serve to confirm placement of the balloon 725 within the treatment area. Treatment areas may range anywhere between the two radiopaque markers 735 within balloon 725, as shown in FIG. 7A.

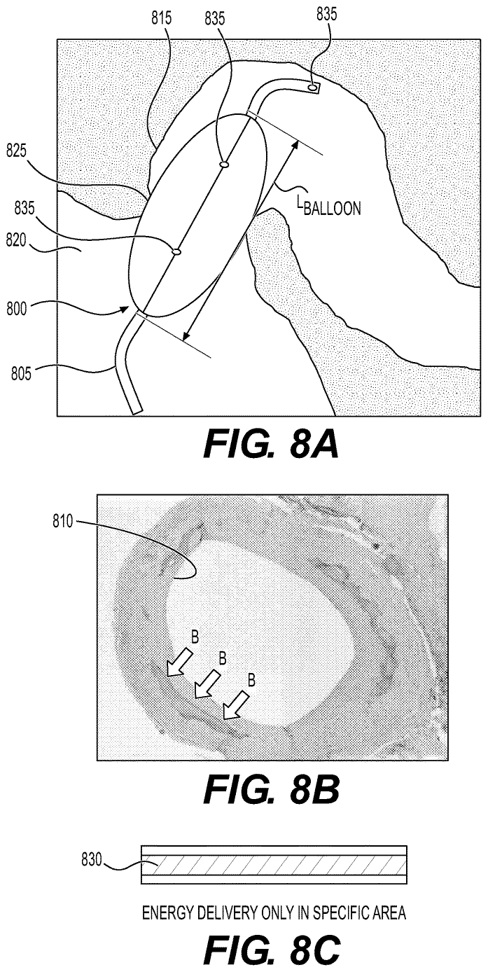

FIGS. 8A, 8B, and 8C show an IVL device 800 according to another embodiment, having a balloon catheter 805, which may be configured for use in an SL 810 of an OA 815, according to another embodiment. In FIG. 8A, the balloon catheter 805 is shown positioned within the SL 810 of the OA 815, which branches off of an ICA 820. As with the embodiment shown in FIGS. 7A, 7B, and 7C, the balloon catheter 805 of this embodiment may have a balloon 825 having a length L_{BALLOON} designed for inflation only in the SL 810 of the OA 815. FIG. 8B shows a cross-sectional view of the SL 810 of the OA 815, as well as arrows B indicating the direction in which the electrical energy from an electrode arrangement 830, shown in FIG. 8C, is delivered to the SL 810. The arrows B in FIG. 8B demonstrate the directional delivery of the electrical energy, as shock waves, and, more specifically, the delivery of the shock waves to a segment of a 360° circumferential area of the SL 810. FIG. 7C is a schematic showing electrical supply from the electrode arrangement 830 of the IVL device 800 to a 360° circumferential area. That is, the balloon catheter 805 of this embodiment is configured to deliver one or more shock waves specifically to the SL 810 in a segment of the 360° circumferential area thereof, while preventing energy from affecting surrounding tissue, such as the ICA 820 or tissue further down in the OA 815. The balloon catheter 805 of this embodiment may also include radiopaque markers 835 within the balloon and at a distal end of the balloon catheter 805, as shown in FIG. 8A, to allow a user (e.g., a surgeon or physician) to properly orient and position the balloon catheter 805 and the balloon 825 prior to providing electrical energy, as part of a method of treatment. In particular, at least one of the radiopaque markers 835 within the balloon 825 may serve to confirm placement of the balloon 825 within the treatment area. Treatment areas may range anywhere between the two radiopaque markers 835 within the balloon 825 along a segment of the 360° circumferential area, as shown in FIG. 8A.

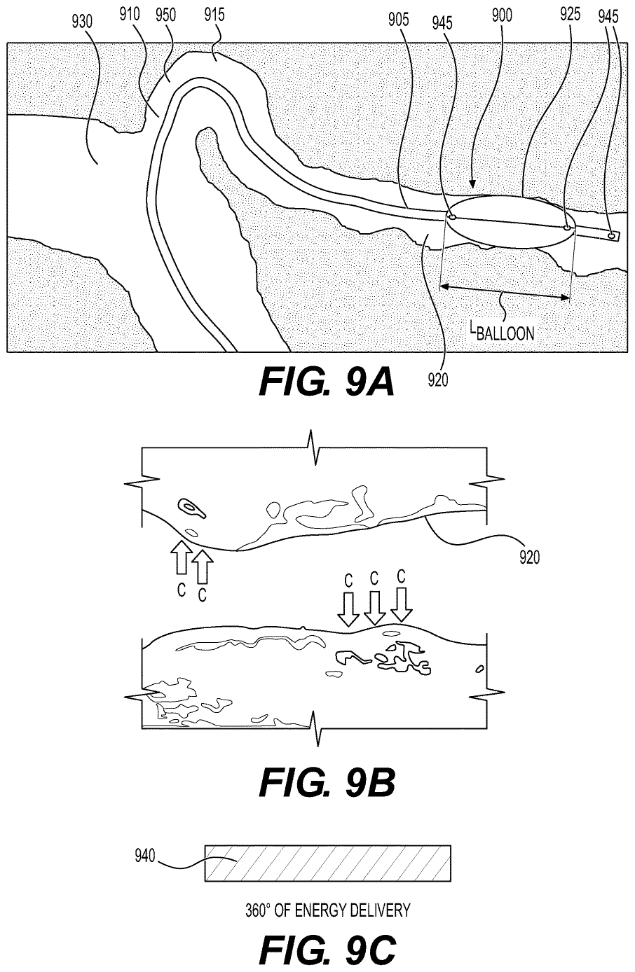

FIGS. 9A, 9B, and 9C show an IVL device 900 according to another embodiment, having a balloon catheter 905, which may be configured for use in a segment 920 of the OA 915 other than the SL 910 (that is, a non-SL segment 920 of the OA 915), according to yet another embodiment. In FIG. 9A, a balloon 925 of the balloon catheter 905 is shown positioned within the non-SL segment 920 of the OA 915, which branches off of an ICA 930. The balloon catheter 905 of this embodiment is configured to deliver one or more shock waves specifically to the non-SL segment 920 of the OA 915 in a 360° circumferential area. The length L_{BALLOON} of the balloon 925 may be optimized to treat a specific length of the non-SL segment 920 of the OA 915. FIG. 9B shows a cross-sectional view of the non-SL segment 920 of the OA 915, as well as arrows C indicating the directions in which the electrical energy from an electrode arrangement 940 of the IVL device 900, shown in FIG. 9C, is delivered to the non-SL segment 920. The arrows C in FIG. 9B demonstrate the delivery of the shock waves to a 360° circumferential area of the non-SL segment 920 of the OA 915. FIG. 9C is a schematic showing electrical supply from the electrode arrangement 940 of the IVL device 900 to a 360° circumferential area. That is, the balloon catheter 905 of this embodiment is configured to deliver one or more shock waves specifically to the non-SL segment 920 in a 360° circumferential area, while preventing energy from affecting surrounding tissue, such as the ICA 930, the SL 910, or other portions of the OA 915. The balloon catheter 905 may also include radiopaque markers 945 within the balloon 925 and at a distal end of the balloon catheter 905, as shown in FIG. 9A, to allow a user (e.g., a surgeon or physician) to properly orient and position the balloon catheter 905 and the balloon 925 prior to providing electrical energy, as part of a method of treatment. In particular, at least one of the radiopaque markers 945 within the balloon 925 may serve to confirm placement of the balloon 925 within the treatment area. Treatment areas may range anywhere between angle ‘a’ 950 of the SL 910 and a distal portion of the OA 915, proximal to the CRA branch (not shown).

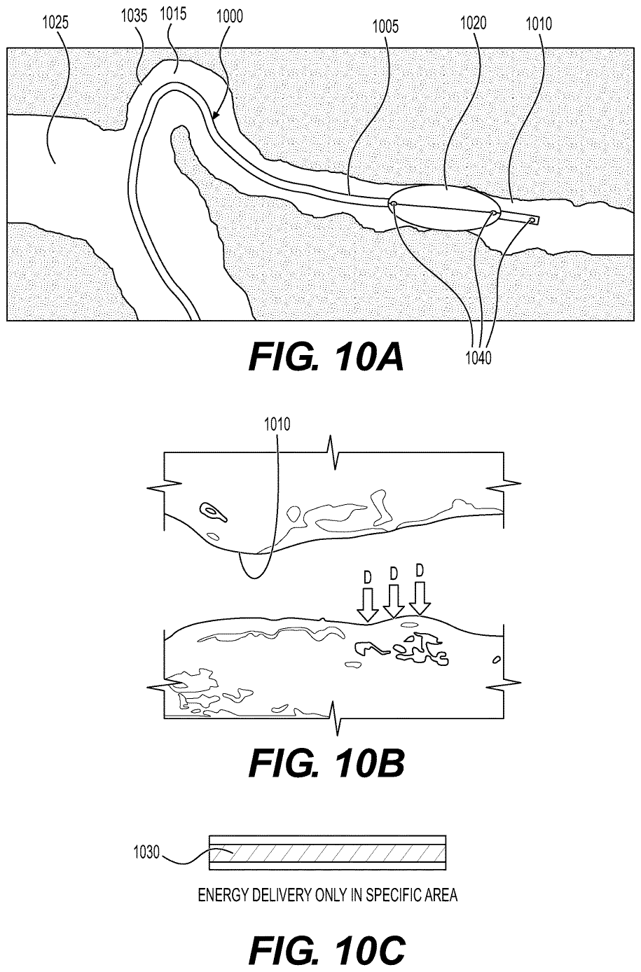

FIGS. 10A, 10B, and 10C show an IVL device 1000 according to another embodiment, having a balloon catheter 1005, which may be configured for use in a non-SL segment 1010 of the OA 1015, according to still another embodiment. In FIG. 10A, a balloon 1020 of the balloon catheter 1005 is shown positioned within the non-SL segment 1010 of the OA 1015, which branches off of an ICA 1025. The balloon catheter 1005 of this embodiment may be configured to deliver one or more shock waves specifically in the non-SL segment 1010 of the OA 1015 in an isolated arc segment of the circumferential area. The length L_{BALLOON} of the balloon 1020 may be optimized to treat a specific length or portion of the non-SL segment 1010 of the OA 1015. FIG. 10B shows a cross-sectional view of the non-SL segment 1010 of the OA 1015, as well as arrows D indicating the direction in which the electrical energy from the electrode arrangement 1030, shown in FIG. 10C, is delivered to the non-SL segment 1010. The arrows D in FIG. 10B demonstrate the delivery of the shock waves to an isolated arc segment of the 360° circumferential area. That is, the balloon catheter 1005 of this embodiment is configured to deliver one or more shock waves specifically to the isolated arc segment of the non-SL segment 1010, while preventing energy from affecting surrounding tissue, such as the ICA 1025, the SL 1035, and other portions of the OA 1015. The balloon catheter 1005 may also include radiopaque markers 1040 within the balloon 1020 and at a distal end of the balloon catheter 1005, as shown in FIG. 10A, to allow a user (e.g., a surgeon or a physician) to properly orient and position the balloon catheter 1005 prior to providing electrical energy, as part of a method of treatment. In particular, at least one of the radiopaque markers 1040 within the balloon 1020 may serve to confirm placement of the balloon 1020 within the treatment area. Treatment areas may range anywhere between angle ‘a’ 1045 of the SL1035, and a distal portion of the OA 1015, proximal to the CRA branch (not shown).

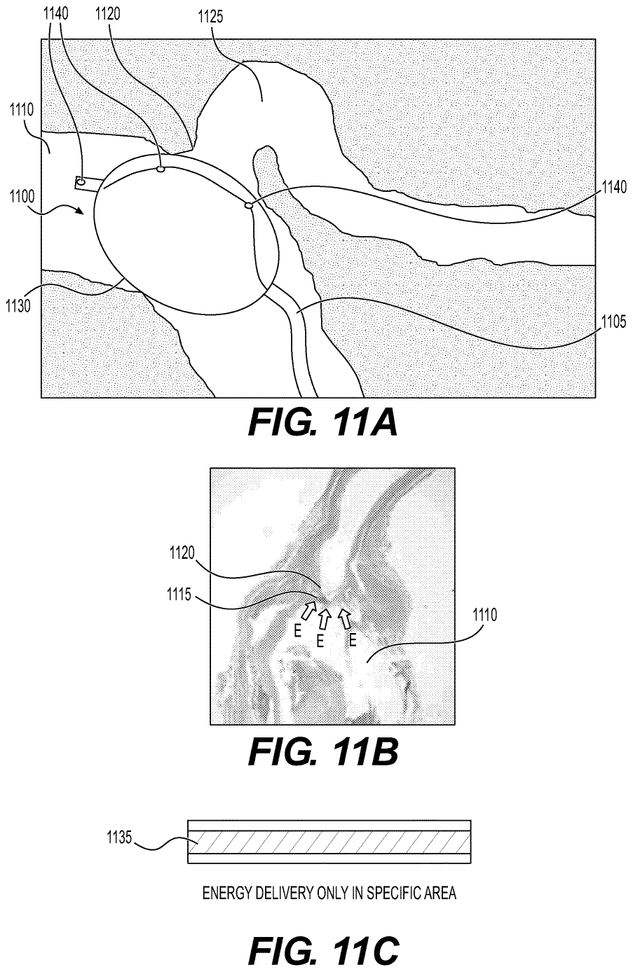

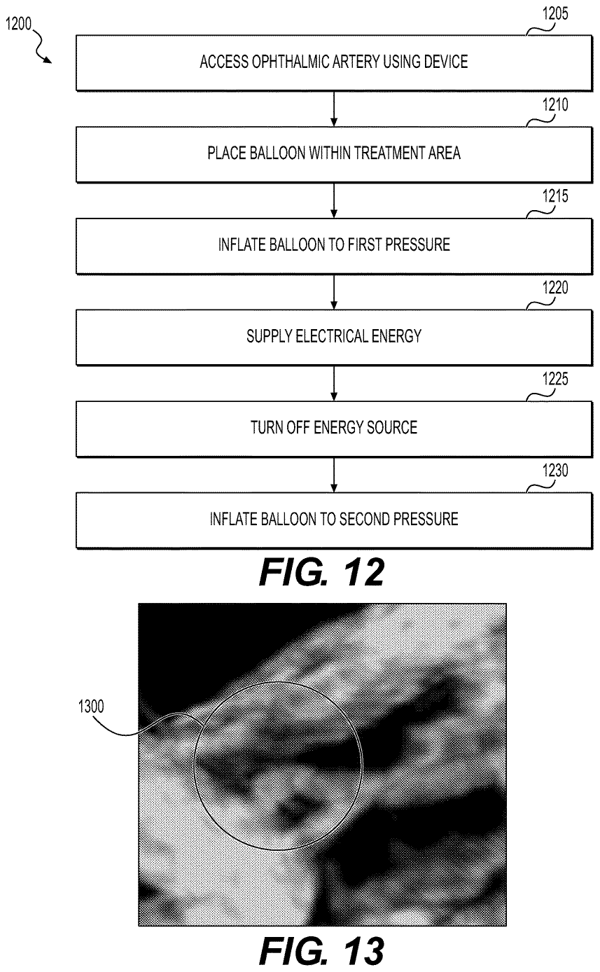

FIGS. 11A, 11B, and 11C show an IVL device 1100 according to another embodiment, having a balloon catheter 1105, which may be configured for use within an ICA 1110 for treating a lesion 1115 (shown in FIG. 11B) in an ostium 1120 between the ICA 1110 and an OA 1125. In FIG. 11A, a balloon 1130 of the balloon catheter 1105 is shown deployed within the ICA 1110 at the ostium 1120. The balloon catheter 1105 of this embodiment may be configured to deliver one or more shock waves to only the ostium 1120. In this embodiment, an electrode arrangement 1135, shown in FIG. 11C, of the balloon catheter 1105 is designed to apply asymmetrical (deflectable) energy delivery along a pattern that conforms to the ostium 1120 of the OA 1125. FIG. 11B shows a cross-sectional view of the ICA 1110, as well as arrows E indicating the direction in which the electrical energy from the electrode arrangement 1135 is delivered to the ICA 1110 at the ostium 1120. FIG. 11C shows the electrode energy delivery to specific portions of a 360° circumferential area of the ICA 1110, and at the ostium 1120. In addition, the balloon catheter of this embodiment may also include radiopaque markers 1140 within the balloon 1130 and at a distal end of the balloon catheter 1105, as shown in FIG. 11A, to allow the user (e.g., the surgeon or physician) to properly orient and position the balloon catheter 1105 prior to providing electrical energy, as part of a method of treatment. In particular, at least one of the radiopaque markers 1140 within the balloon 1130 may serve to confirm placement of the balloon 1130 within the treatment area. Use of the balloon catheter 1105 of this embodiment may also allow for continuous blood flow through the balloon 1130, when inflated, within the ICA 1110 (e.g., use of a toroidal balloon and/or use of a balloon having side holes-not shown).

Electrode arrangements may include conventional or low profile electrodes to enable a minimum balloon crossing profile design. In addition or alternatively, the shock wave energy source may have energy control that allows for some variation in how energy is delivered to the balloon. Specifically, these energy control delivery variations include controls for the number of pulses, single strength, variable strength, time sequenced, or reversed polarity, each of which can be adjusted depending on target anatomy (i.e., ostium, short limb, long limb). The shock waves may include forward directed, radial, or circumferentially directed shock waves, or any combination of directions, as well as asymmetrical patterns of the same. In addition, the balloon may be coated with a drug to be delivered after the shock wave therapy has been accomplished. In this case, there may be multiple stages of energy delivery prior to drug delivery. Lastly, the balloon design may include a non-cavitation balloon to provide therapeutic shock wave energy within a small diameter artery.

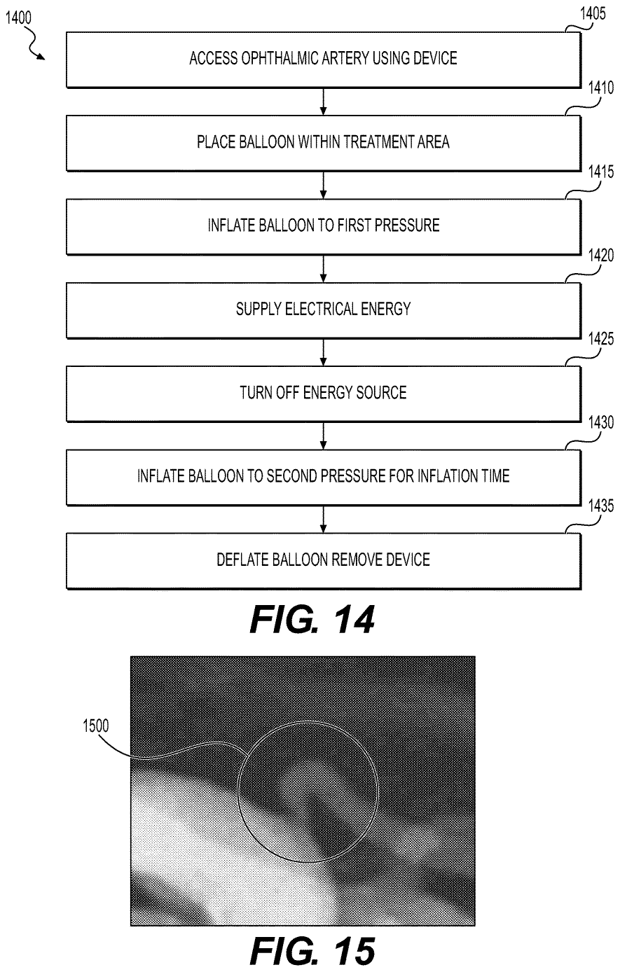

FIG. 12 is a flowchart of a method 1200 of using an IVL device, according to one or more embodiments. In step 1205, a microcatheter and conventional GW of the IVL device may be used to access to the OA via the ICA. Once GW purchase (i.e., a firm hold) within the OA is established, in step 1210, a balloon catheter of the IVL device may be placed within the OA, at the desired anatomical location or treatment area (e.g., within the ostium, the short limb, or the long limb). The balloon may then be inflated to a first pressure, in step 1215, so that it is firmly apposed to the treatment area. Then, in step 1220, electrical energy may be supplied, so that one or more shock waves are initiated to provide therapy to the treatment area at a predetermined energy level and/or for a predetermined period of time. After the energy is delivered, in step 1225, the energy source may be turned off, and in step 1230, the balloon may be inflated to a second pressure, which may be the same or a greater pressure, to provide angioplasty of the OA. In some embodiments, the method may further include exchanging the balloon for another angioplasty specific balloon to continue with angioplasty. FIG. 13 is a Cone Beam Computed Tomography (CB CT) image of a calcified OA SL lesion, shown in circle 1300, which may be a target of an IVL balloon catheter treatment process, as in method 1200. Although the method 1200 is described as including steps 1205 to 1230, the method 1200 may include a subset of these steps or additional steps. For example, the method 1200 may include step 1205 of accessing the OA using the microcatheter and GW of the device, step 1210 of placing the balloon catheter within the OA, step 1215 of inflating the balloon, step 1220 of supplying electrical energy to initiate one or more shock waves, and step 1230 of inflating the balloon again to provide angioplasty of the OA.

FIG. 14 is a flowchart of another embodiment of a method 1400 of use of an IVL device, according to one or more embodiments. In step 1405, a microcatheter and a conventional GW of the IVL device may be used to gain access to an OA via an ICA. Once GW purchase (i.e., a firm hold) is established, in step 1410, a balloon catheter of the IVL device may be placed within the OA, such that it is at the desired anatomical location or treatment area (e.g., within the ostium, the short limb, the long limb, or some other target area). In some embodiments, the desired anatomical location may be within a vessel segment with an eccentric cross section, and which has a lesion therein. Once in location, in step 1415, a balloon of the balloon microcatheter may then be inflated to a first pressure, so that it is in firm apposition to the treatment area. Then, in step 1420, electrical energy may be supplied, so that one or more shock waves may be initiated to provide therapy to the treatment area at a predetermined energy level, in a predetermined pattern, and for a predetermined period of time to a targeted area of the lesion. After the energy is delivered, in step 1425, the energy source may be turned off and, in step 1430, the balloon may be inflated to a second pressure, which may be the same as or greater than the first pressure, to provide angioplasty of the vessel segment (e.g., an artery), so that the eccentricity effect on dilatation is minimized. The period of time in which the balloon remains inflated to the second pressure may be referred to as inflation time. Once the inflation time is complete, in step 1435, the balloon may be deflated and removed. Although the method 1400 is described as including steps 1405 to 1435, the method 1400 may include a subset of these steps or additional steps. FIG. 15 is a Digital Subtraction Angiography (DSA) image of a non-concentric OA SL lesion 1500, which may be a target of the method 1400.

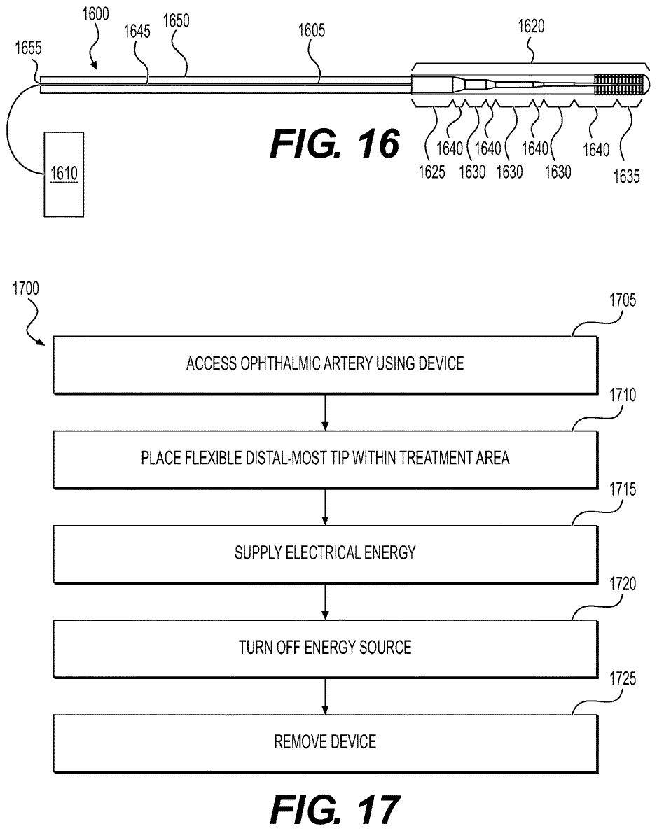

FIG. 16 is a schematic of an IVL device 1600, according to one embodiment, which may include a GW 1605, a shock wave generator 1610 with an electrode arrangement (single or multiple, monopolar or bipolar) and a power source (not shown). The IVL device 1600 may be constructed to provide a minimal crossing profile, as well as flexibility, torqueability, and pushability to access an OA. In addition, the IVL device 1600 may be configured for use in conjunction with an appropriately sized microcatheter. The IVL device 1600 of this embodiment utilizes the capability of the GW 1605 to navigate small, tortuous anatomy. As one example of such use, the GW 1605 of the IVL device 1600 is designed to fit within the diameter of the OA, as noted above, and is configured to deliver a therapeutic shock wave to the target anatomy. The energy ranges of the IVL device 1600 may be optimized to treat the small diameter of the OA.

The GW 1605 may be constructed to be steerable and extremely flexible with a multi tapered corewire tip design that provides a range of tip stiffnesses (e.g., standard, soft, or super soft). Specifically, the GW may have a tapered tip portion 1620, which may include a proximal end portion 1625, one or more intermediate portions 1630, and a distal end portion 1635. The GW 1605 may also have tapered intermediate portions 1640 between each of the proximal end portion 1625, the one or more intermediate portions 1630, and the distal end portion 1635. The diameters of these portions of the GW 1605 may be in range of 0.010 inch (0.254 mm) to 0.038 inch (0.965 mm). The length of the GW 1605 may be in a range of 39.37 inches (100 cm) to 125.98 inches (320 cm) (also referred to as exchange length). In some embodiments, the GW 1605 may be a triple tapered corewire with a diameter along the tapered tip portion 1620 (which may include at least one intermediate portion 1630, at least one tapered intermediate portion 1640, and the distal end portion 1635) being about 0.014 inch (0.356 mm), which after some length, tapers to 0.012 inch (0.305 mm), and again after some length, tapers to 0.010 inch (0.254 mm) at the distal end portion 1635 of the GW 1605. The tapered tip portion 1620 may also be configured to be shapeable. This GW 1605 having the tapered tip portion 1620 with a combination of tapers and diameters facilitates navigation, access, and purchase of the GW 1605 into the OA and movement to the desired treatment location. A proximal portion 1645 of the GW 1605 that contains an electrode (not shown) is housed within a hypotube 1650, which begins at the tapered tip portion 1620 and extends in a proximal direction to a proximal-most end 1655 of the GW 1605, as shown in FIG. 16. The hypotube 1650 may be formed of a material that is metallic, non-metallic, or a combination thereof. The shock wave may be directed through the hypotube 1650, that is, through the walls of the hypotube 1650, or through a window in the hypotube 1650 (not shown). As noted above, the shock wave may be deliverable in a 360° circumferential area or a segment of the 360° circumferential area.

FIG. 17 is a flowchart of a method 1700 of using an IVL device having a microcatheter with a non-removable corewire and a GW with a flexible distal-most tip, according to one embodiment. In step 1705, the microcatheter and GW may be used to gain access to an OA via an ICA. Once GW purchase (i.e., a firm hold) is established, in step 1710, the GW may be placed within the OA, such that the flexible, distal-most tip of the device is at the desired anatomical location or treatment area. Then, in step 1715, electrical energy may be supplied, so that one or more shock waves are initiated to provide therapy at a predetermined energy level and for a predetermined period of time. After the energy is delivered, in step 1720, the energy source is turned off and in step 1725, the microcatheter and the GW are removed. Although the method 1700 is described as including steps 1705 to 1720, the method 1700 may include a subset of these steps or additional steps. For example, the microcatheter may be left in place within the OA, while the GW is removed.

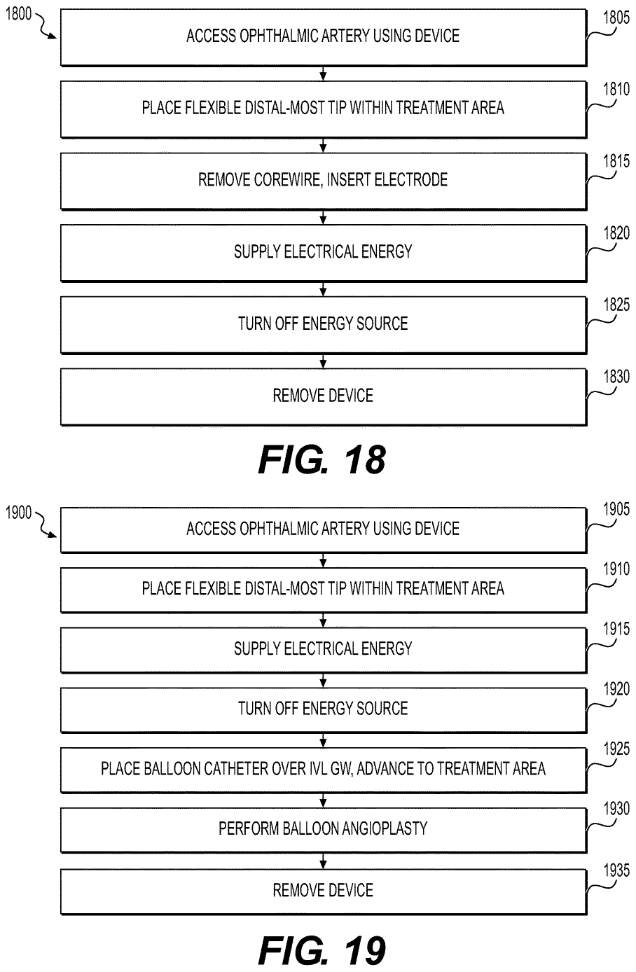

FIG. 18 is a flowchart of a method 1800 of using an IVL device that includes a microcatheter with a corewire and a removable GW, according to still another embodiment. The method 1800 may include a step 1805 of using the microcatheter and GW to gain access to the OA via the ICA, and, once GW purchase (i.e., a firm hold) is established, a step 1810 of placing the GW in within the OA, such that the GW is at the desired anatomical location with the flexible most distal tip being distal to the treatment area. Then, in step 1815, the corewire is removed and replaced with an electrode. In step 1820, electrical energy may be supplied, so that one or more shock waves are initiated to provide therapy at a predetermined energy level and for a predetermined period of time. After the energy is delivered, in step 1825, the energy source is turned off and in step 1830, the microcatheter and the GW are removed. Although the method 1800 is described as including steps 1805 to 1830, the method 1800 may include a subset of these steps or additional steps.

FIG. 19 is a flowchart of a method 1900 of using an IVL device that includes a microcather and a GW, according to yet another embodiment. In step 1905, the microcatheter and GW are used to gain access to the OA via the ICA. Once GW purchase (i.e., a firm hold) is established, in step 1910, the GW is placed within the OA, such that it is at the desired anatomical location with the flexible most distal tip distal to the treatment area. Then, in step 1915, electrical energy may be supplied, so that one or more shock waves are initiated to provide therapy at a predetermined energy level and for a predetermined period of time. After the energy is delivered, in step 1920, the energy source is turned off and in step 1925, a balloon catheter is placed over the IVL GW and advanced to the desired anatomical location or treatment area. Then, in step 1930, balloon angioplasty is performed and in step 1935, the balloon, the microcatheter, and the GW are removed. Although the method 1900 is described as including steps 1905 to 1935, the method 1900 may include a subset of these steps or additional steps.

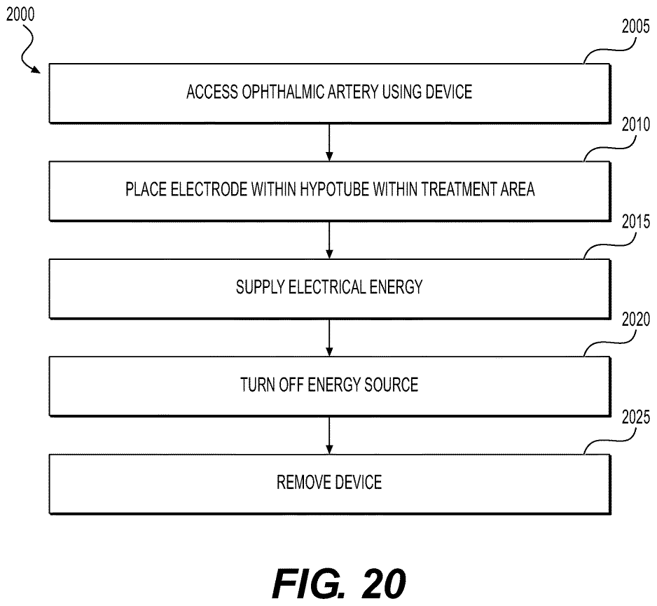

FIG. 20 is a flowchart of a method 2000 of using an IVL device having a microcatheter and a GW, according to another embodiment. The GW used in the method 2000 has an electrode housed within a hypotube, and the hypotube is capable of being filled with saline to allow for the generation of a shock wave within the contained saline. In step 2005, the microcatheter and GW are used to gain access to the OA via the ICA. Once GW purchase (i.e., a firm hold) is established, in step 2010, the GW is placed within the OA such that it is at the desired anatomical location or treatment area. Then, in step 2015, electrical energy is supplied, such that one or more shock waves are initiated and penetrate the hypotube, thereby providing treatment to the adjacent tissue at the treatment area. After the energy is delivered, in step 2020, the energy source is turned off, and in step 2025, the microcather and GW are removed. In some embodiments, the electrode may be housed within the hypotube such that the hypotube is permeable, and so that blood is allowed to flow into the hypotube. In this case, the one or more shock waves may be generated within the blood contained within the hypotube. The shock waves are capable of penetrating the permeable hypotube and providing treatment to the adjacent tissue. Although the method 2000 is described as including steps 2005 to 2025, the method 2000 may include a subset of these steps or additional steps.

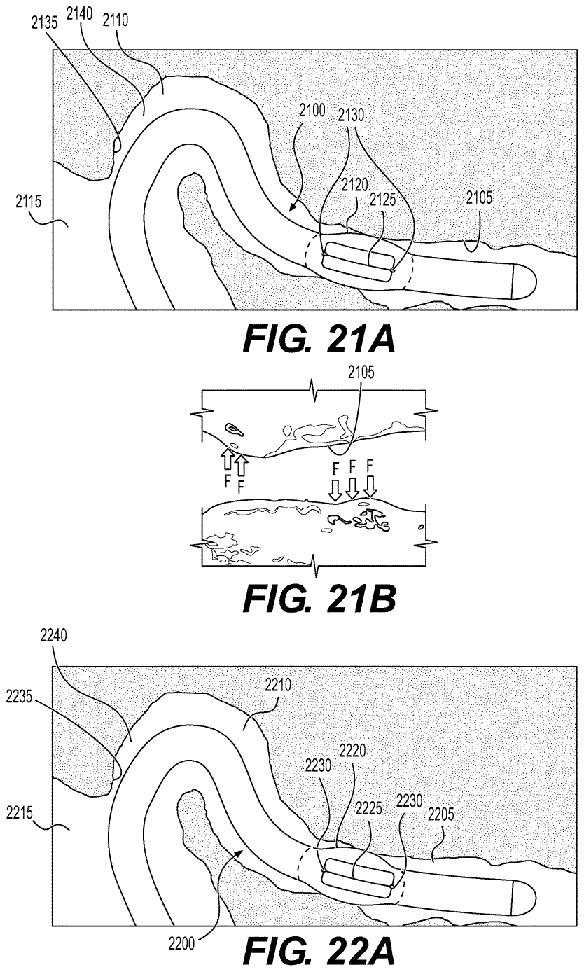

FIGS. 21A and 21B show an IVL device 2100 that may be configured for use in a non-SL segment 2105 of an OA 2110, according to one embodiment. Specifically, FIG. 21A shows the IVL device 2100, an ICA 2115, the OA 2110, and the non-SL segment 2105 of the OA 2110, and a balloon 2120 and an electrode 2125 of the IVL device 2100 being positioned within the non-SL segment 2105 of the OA 2110. FIG. 21B shows a cross-sectional view of the non-SL segment 2105, and arrows F indicating the directions in which electrical energy, in the form of shockwaves, are delivered to the 360° circumferential area of the non-SL segment 2105. That is, in this embodiment, the IVL device 2100 may provide shock wave delivery to the non-SL segment 2105 of the OA 2110 in a 360° circumferential area, as shown in FIG. 21B. The length of the electrode 2125 may be optimized to treat a specific length of the non-SL segment 2105 of the OA 2110 and may be asymmetrical (deflectable). The IVL device 2100 of this embodiment may also include radiopaque markers 2130 to allow a user (e.g., a surgeon or a physician) to properly orient the IVL device 2100 prior to providing treatment. In particular, at least one of the radiopaque markers 2130 within the balloon 2120 may serve to confirm placement of the balloon 2120 within the treatment area. Treatment areas may range anywhere between an angle ‘a’ 2135 of the SL 2140 and a distal end of the OA 2110 proximal to the CRA branch. In FIG. 21A, a portion of the IVL device 2100 is cut-away to show the electrode 2125 therein.

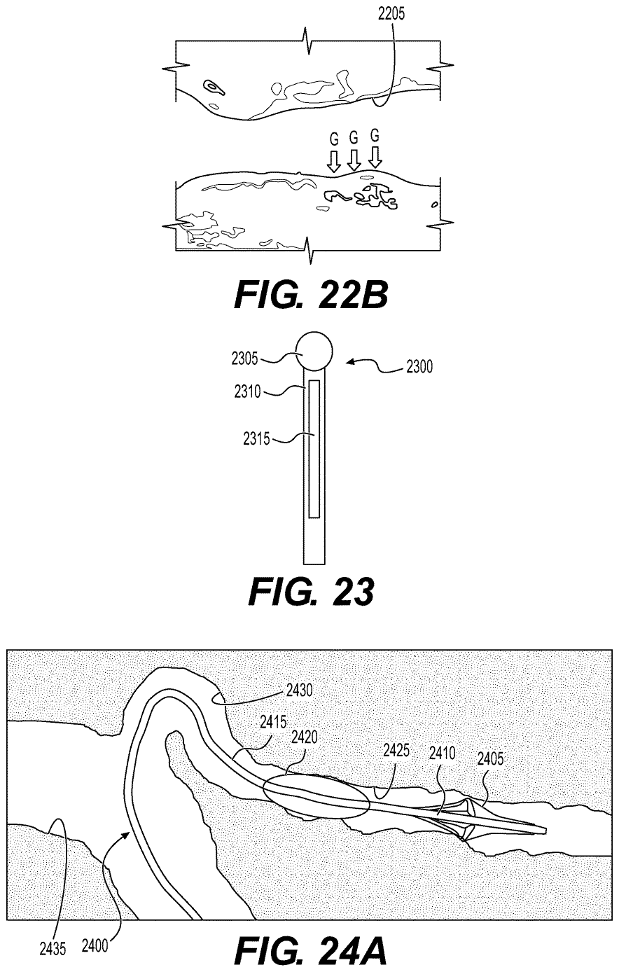

FIGS. 22A and 22B show an IVL device 2200 that may be configured for use in a non-SL segment 2205 of an OA 2210, according to another embodiment. Specifically, FIG. 22A the IVL device 2200, an ICA 2215, the OA 2210, and the non-SL segment 2205 of the OA 2210, and a balloon 2220 and an electrode 2225 of the IVL device 2100 being positioned within the non-SL segment 2105 of the OA 2110. FIG. 22B shows a cross-sectional view of the non-SL segment 2205, and arrows G indicating the direction in which electrical energy, in the form of shockwaves, are delivered to an isolated arc segment of the 360° circumferential area of the non-SL segment 2205. That is, in this embodiment, the IVL device 2200 may provide shock wave delivery to the non-SL segment 2205 of the OA 2210 in an isolated arc segment of the 360° circumferential area, as shown in FIG. 22B. The length of the electrode 2225 may be optimized to treat a specific length of the OA 2210, and may be asymmetrical (deflectable). The IVL device 2200 may also include radiopaque markers 2230 to allow a user (e.g., a surgeon or a physician) to properly orient the IVL device 2200 prior to providing treatment. In particular, at least one of the radiopaque markers 2230 within the balloon 2220 may serve to confirm placement of the balloon 2220 within the treatment area. Treatment areas may range anywhere between an angle ‘a’ 2235 of an SL 2240 and a distal end of the OA 2210 proximal to the CRA branch. In FIG. 22A, a portion of the IVL device 2200 is cut-away to show the electrode 2225 therein.

FIG. 23 shows an end view of an electrode 2305 of an IVL device 2300 with a portion 2310 having a coating thereon, and another portion 2315 not having a coating, according to one or more embodiments. In one or more embodiments, the electrode 2305 may be coated with an insulative material so as to direct the arc of electrical energy in a specific direction. These coatings may include insulative materials, such as diamond like coating (DLC), titanium nitride (TiN), or other materials well suited to providing electrical isolation. The pattern may be simple or complex, depending on the application. These particular coatings may be used with mono or bipolar electrodes and may be used to both provide general insulative properties as well as directing the arc of electrical energy so that the shock wave may be focused at a particular vascular target. The coatings may also provide for a consumable use in a similar manner as the electrode 2305.

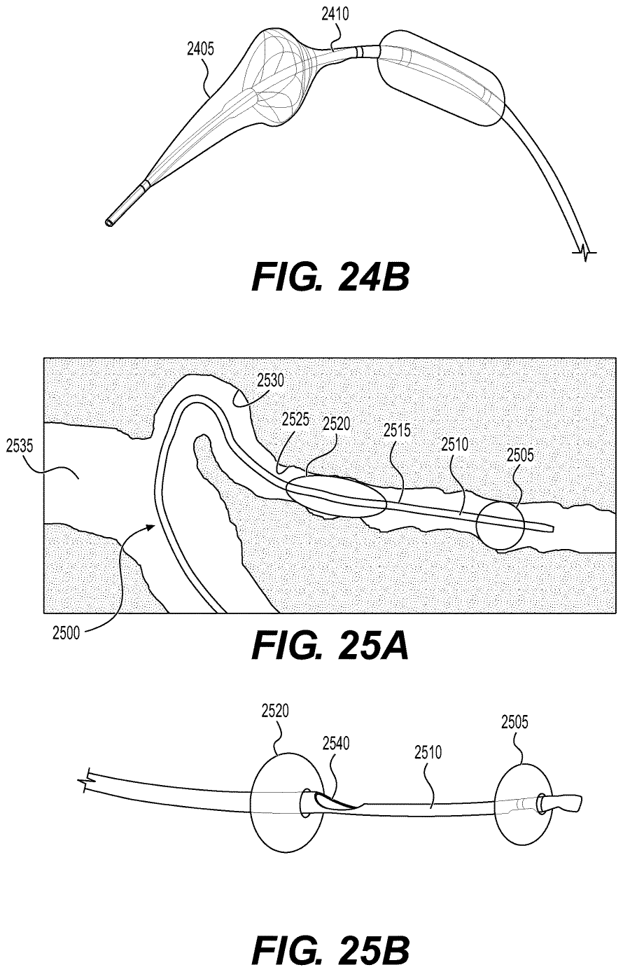

In one or more embodiments in which the IVL device is configured for use in either the SL or non-SL segments of the OA or within the ICA, the IVL device may also include a portion that provides distal protection, such as a distal filter. As one example, FIGS. 24A and 24B show an IVL device 2400 having a distal filter 2405 on a distal segment 2410 of the a balloon catheter 2415 of the IVL device 2400. FIG. 24A is a schematic showing placement of a balloon 2420 of the IVL device 2400 within a non-SL segment 2425 of an OA 2430, which branches off from an ICA 2435. FIG. 24B is a detail view of the distal segment 2410 of the IVL device 2400, including the distal filter 2405. The distal filter 2405 is configured to be an integral part of the IVL device 2400 and, in use, may be deployed (that is, it may be deployable) prior to performing a procedure, such as a lithotripsy, an angioplasty, or a lithotripsy and a subsequent angioplasty, to catch debris resulting from the procedure. In particular, as one example, in use, once angioplasty is complete, the balloon 2420 may be deflated, the distal filter 2405 may be closed (that is, it may be closable or retractable, capturing any debris), and the IVL device 2400 may be removed from the patient. This arrangement protects the other vasculature and portions of the eye from debris generated during angioplasty of the OA 2430. The distal filter 2405 may be provided on any one of the IVL devices described herein, such as the IVL devices configured for use in the SL of the OA or within the ICA. In addition, as in the embodiments described above, one or more radiopaque markers (not shown) may be included, to allow a user (e.g., a surgeon or a physician) to properly orient the IVL device 2400 prior to providing treatment, including at least one radiopaque marker within the balloon 2420 may serve to confirm placement of the balloon 2420 within the treatment area.

In one or more embodiments in which the IVL device is configured for use in either the SL or non-SL segments of the OA or within the ICA, the IVL device may also include a distal occlusion balloon, as a means of distal protection. As shown in FIGS. 25A and 25B, for example, an IVL device 2500 may include a distal occlusion balloon 2505, on a distal segment 2510 of the a balloon catheter 2515 of the IVL device 2500. FIG. 25A is a schematic showing placement of a balloon 2520 of the IVL device 2500 within a non-SL segment 2525 of an OA 2530, which branches off from an ICA 2535. FIG. 25B is a detail view of the distal segment 2510 of the IVL device 2500, including the distal occlusion balloon 2505. The distal occlusion balloon 2505 may have an aspiration lumen] 2540, which provides aspiration capability, and which may be provided between the two balloons (that is, between the IVL balloon 2520 and the distal occlusion balloon 2505), as shown in FIGS. 25A and 25B. Although this configuration has been used in carotid artery stenting to provide proximal and distal protection, in applications for treatment of the OA (e.g., angioplasty of the OA), a proximal protection balloon has not been needed, as the angioplasty balloon may serve this purpose. As an example, in use, the distal occlusion balloon 2505 of the IVL device 2500 may be deployed prior to lithotripsy and angioplasty of the OA 2530. Once angioplasty is complete, aspiration may be performed, removing any embolic debris from the occluded OA artery segment. When embolic debris removal is completed, the distal occlusion balloon 2505 may be deflated, and the IVL device 2500 may be removed from the patient. The distal occlusion balloon 2505 and [insert name of aspiration structure] 2540 may be provided on any one of the IVL devices described herein, such as the IVL devices for use in the SL of the OA or the ICA. In addition, as in the embodiments described above, one or more radiopaque markers (not shown) may be included, to allow a user (e.g., a surgeon or a physician) to properly orient the IVL device 2500 prior to providing treatment, including at least one radiopaque marker within the balloon 2520 may serve to confirm placement of the balloon 2520 within the treatment area.

The embodiments of the IVL devices and the related methods described herein may provide for treatment of the OA for eye disease, including angioplasty of the OA, using a combination of IVL with balloon angioplasty and GWs, which may improve visual acuity in patients, such as patients with AMD. The IVL devices and related methods may also provide for treatment of calcified lesions to improve angioplasty outcomes. Further, the IVL devices and related methods of this disclosure may minimize the potential for embolic events in the retinal circulation, as a result of the OA angioplasty.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. An intra vascular lithotripsy (IVL) device for treatment of lesions and/or calcifications within vasculature behind an eye of a subject, the IVL device comprising:
a guidewire configured to access the vasculature of the subject; and
a balloon catheter including:
an inflatable balloon configured to be placed within a treatment area of the vasculature of the subject and to be inflated therein, and
an electrode arrangement within the balloon,
wherein, the electrode arrangement is configured to deliver one or more shock waves, and to the treatment area, when electrical energy is supplied to the electrode arrangement.

2. The IVL device of claim 1, wherein the electrode arrangement is configured to deliver the one or more shock waves in multiple directions, around a 360° circumferential area thereof.

3. The IVL device of claim 1, wherein the electrode arrangement is configured to deliver the one or more shock waves in one direction, to a segment of a 360° circumferential area thereof.

4. The IVL device of claim 1, further comprising one or more radiopaque markers, including at least one radiopaque marker within the balloon, and configured to confirm placement of the balloon within the treatment area of the vasculature of the subject.

5. The IVL device of claim 4, wherein the one or more radiopaque markers include at least two radiopaque markers within the balloon, and at least one radiopaque marker at a distal end of the balloon catheter.

6. The IVL device of claim 1, wherein a diameter of the inflated balloon is in a range of about 0.75 mm to about 2.5 mm.

7. The IVL device of claim 1, wherein a length of the balloon when inflated is in a range of about 4 mm to about 30 mm.

8. The IVL device of claim 1, further comprising a distal filter provided on a distal end of the balloon catheter, the distal filter being deployable to catch debris resulting from a procedure.

9. A method of treating lesions and/or calcifications within vasculature behind an eye of a subject using an intra vascular lithotripsy (IVL) device, the IVL device comprising a guidewire for accessing vasculature of the subject, a balloon catheter including an inflatable balloon configured to be placed within a treatment area of the vasculature of the subject and to be inflated therein, and an electrode arrangement within the balloon, the method comprising:
accessing an ophthalmic artery (OA) of the subject using the guidewire;
placing the balloon within a treatment area near an internal carotid artery (ICA) or within the OA;
inflating the balloon a first time within the treatment area; and
supplying electrical energy to the electrode arrangement, thereby initiating and delivering one or more shock waves to the treatment area.

10. The method of claim 9, further comprising:
deflating the balloon following the angioplasty; and
removing the IVL device from the vasculature of the subject.

11. The method of claim 9, further comprising receiving information from a feedback loop regarding an effectiveness of the delivered one or more shock waves.

12. The method of claim 9, wherein the one or more shock waves include one or more of forward directed waves, radially directed waves, circumferentially directed waves, or partially arc directed waves, or combinations thereof.

13. The method of claim 9, wherein the electrode arrangement is configured to deliver the one or more shock waves in multiple directions, around a 360° circumferential area thereof.

14. The method of claim 9, wherein the electrode arrangement is configured to deliver the one or more shock waves in one direction, to a segment of a 360° circumferential area thereof.

15. The method of claim 9, wherein the IVL device further comprises one or more radiopaque markers, including at least one radiopaque marker within the balloon, and configured to permit a user to confirm placement of the balloon within the treatment area of the vasculature of the subject.

16. The method of claim 15, wherein the one or more radiopaque markers include at least two radiopaque markers within the balloon, and at least one radiopaque marker at a distal end of the balloon catheter.

17. The method of claim 9, wherein a diameter of the inflated balloon is in a range of about 0.75 mm to about 2.5 mm.

18. The method of claim 9, wherein a length of the inflated balloon is in a range of about 4 mm to about 30 mm.

19. The method of claim 9, wherein the IVL device further comprises a distal filter provided on a distal end of the balloon catheter, and the method further comprises deploying the distal filter to catch debris before performing the angioplasty, to catch debris.

20. An intra vascular lithotripsy (IVL) device for treatment of lesions and/or calcifications within vasculature behind an eye of a subject, the IVL device comprising:
a hypotube;
a guidewire configured to access the vasculature of the subject, a proximal portion of the guidewire being housed within the hypotube; and
an electrode arrangement within the guidewire,
wherein, the electrode arrangement is configured to deliver one or more shock waves, through the guidewire and the hypotube, and to a treatment area, when electrical energy is supplied to the electrode arrangement.
